(19) United States
(12) Patent Application Publication
(10) Pub. No.: US 2006/0254000 A1
(43) Pub. Date: Nov. 16, 2006

(54) Composition for permanent dyeing of keratinous fibers

(76) Inventors: Gerard Ducheron, Brie Sur Marne (FR); Hélène Jauffret, Asnieres Sur Seine (FR)

Correspondence Address: YOUNG & THOMPSON, 745 SOUTH 23RD STREET, 2ND FLOOR, ARLINGTON, VA, 22202, US

(21) Appl. No.: 10/542,273

(22) PCT Filed: Jan. 14, 2004
(86) PCT No.: PCT/FR04/50015

§ 371 Date: May 16, 2006

(30) Foreign Application Priority Data
Jan. 15, 2003 (FR) 03 00409

(51) Int. Cl. A61K 8/00

(52) U.S. Cl. 8/405

(57) ABSTRACT

A composition for the permanent dyeing of keratinous fibres, includes at least one dye precursor, at least one alkaline agent, and at least one oxidizing compound, and further includes: a protein hydrolyzate, a quaternized copolymer of di-methylallyl ammonium and acrylic acid, a N-methyl triethanolamine methylsulphate di-alkyl ester, and a di-alkyl carbonate.

The object of the present invention is to provide a cosmetic composition for permanent dyeing of keratinous fibres, and in particular, of hair fibres.

Such a composition has the property of alleviating the deleterious effects of the chemical method implemented upon a permanent dyeing of hair fibres and of holding upon the dyeing step the hair natural properties.

The only hair dyeing methods able to perfectly and durably cover hair are oxidative dyeing methods resulting in the so-called permanent dyes.

Such methods implement the combined action of an oxidizing agent, in general hydrogen peroxide, and an alkaline base, preferably aqueous ammonia. Such a combination has the effet to simultaneously induce, the bleaching of hair natural or artificial pigmentation, and the oxidation of dye precursors, resulting in creating a new dyed shade.

The alkaline base, preferably aqueous ammonia, facilitates the dissolution of dyeing agents and, through the alkanization of the medium, enhances the bleaching action of hydrogen peroxide, taking part in the release of the active oxygen.

Aqueous ammonia can also act on the fibre swelling in causing scales to open. It thereby facilitates the penetration of dye precursors and ensures a homogenous distribution of the pigments up to the fibre core.

The oxidizing agent, generally hydrogen peroxide, has two functions: bleaching the existing pigments so as to avoid dye variations resulting from the hair initial colour and triggering the oxidative process of the dye precursors. In a first step, such precursors are transformed into very reactive intermediates, which couple therebetween to form, in a second oxidative condensation step, coloured copolymers able to securely be fixed in the keratinous fibre.

Such a permanent dyeing technique gives excellent results, both regarding the white hair cover (close to 100%), the shade variety being offered and the relative dye durability over time.

It however has several inconvenients, such as quickly causing large physico-chemical damage on the fibre.

Because of the steady growth of the fibres in their original natural shade, resulting in no aesthetic boundary with the artificially dyed lengths, and the progressive dye removal, dye regular applications are required.

Subject to the extreme alkaline-oxidizing conditions implemented during the process, the fibre, chemically and physically altered in its entirety, progressively losses its natural properties. Inevitably, over dyeing operations, hair become dry and harsh to the touch and loses some of their natural flexibility and brightness. The removal resistance of the dye decreases. Thereby, dyeing products seem to lack performance.

The Applicant found out that adding a specific quaternary complex to the commonly used dyeing bases makes it possible to durably and efficiently dye hair (improved dye durability), while holding their natural mechanical properties (resistance and flexibility) and improving their cosmetic properties (softness and brightness).

The present invention has therefore as an object a composition for the permanent dyeing of keratinous fibres, and in particular, of hair, comprising at least one oxidizing compound, at least one dye precursor and at least one alkaline agent, characterized in that it further comprises
a protein hydrolyzate,
a quaternized copolymer of di-methylallyl ammonium and acrylic acid,
a N-methyl triethanolamine methylsulphate di-alkyl ester, and
a di-alkyl carbonate.

Advantageously, the protein hydrolyzate, the quaternized copolymer, the N-methyl triethanolamine methylsulphate di-alkyl ester and the di-alkyl carbonate form a quaternary associative complex within the composition.

Preferably, the protein hydrolyzate is a sericin hydrolysate, a silk protein.

The sericin hydrolyzate is a concentrated and purified mixture of peptides with a molecular size lower than or equal to 20 kDa. It is characterized by a high serine and aspartic acid content. Ideally, it could be comprised of 18 amino acids, most of which have highly polar side groups such as hydroxy, carboxy and amino groups.

The protein hydrolyzate is preferably present in an amount ranging from 0.1 to 10%, more preferably from 1 to 5% in weight based on the total weight of the composition.

The quaternized copolymer of di-methylallyl ammonium and acrylic acid included in the composition according to the invention generally meets the following formula I: 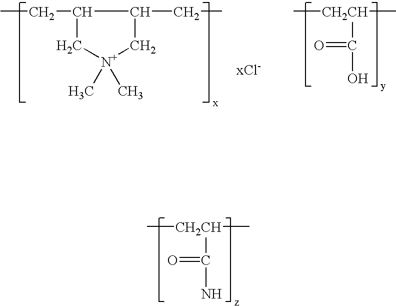
wherein x ranges from 1 to 1,000, preferably from 1 to 100, and y and z independently range from 0 to 1,000, preferably from 0 to 100.

Advantageously, the quaternized copolymer is a polyquaternium, for example a polyquaternium 22 (CTFA name).

The quaternized copolymer of di-methylallyl ammonium and acrylic acid preferably represents from 0.1 to 1.5%, more preferably from 0.5 to 1%, in weight based on the total weight of the composition.

The N-methyl triethanolamine methylsulphate di-alkyl ester included in the composition according to the invention generally meets the following formula II: 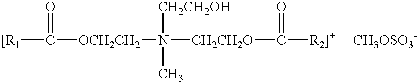
wherein R₁ and R₂ are Cn alkyl or alkenyl, where n ranges from 1 to 100, preferably from 6 to 20.

The alkyl groups useful for R₁ and R₂ could be caproyl, caprylyl, capryl, lauryl, myristyl, cetyl, stearyl, arachidyl or behenyl chains. The alkenyl groups useful for R₁ and R₂ could be caproleyl, lauroleyl, myristoleyl, palmitoleyl, oleyl, linoleyl, linolenyl, arachidonyle or erucyl chains.

Advantageously R₁ and R₂ represent an alkyl group derived from coconut oil, in particular a lauryl chain. Hence, the di-alkyl ester to be preferably used is a N methyl triethanolamine methylsulphate dicocoyl ester.

The N-methyl triethanolamine methylsulphate di-alkyl ester preferably represents from 0.1 to 5%, more preferably from 0.5 to 2%, in weight based on the total weight of the composition.

The di-alkyl carbonate included in the composition according to the invention generally meets the following formula III: 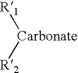
wherein R′₁, and R′₂ are Cₙ, straight or branched, alkyl or alkenyl groups, where n ranges from 1 to 100, preferably from 6 to 20.

The alkyl groups useful for R′₁ and R′₂ could be caproyl, caprilyl, capryl, lauryl, myristyl, cetyl, stearyl, arachidyl or behenyl chains. The alkenyl groups useful for R′₁, and R′₂ could be caproleyl, lauroleyl, myristoleyl, palmitoleyl, oleyl, linoleyl, linolenyl, arachidonyle or erucyl chains.

Advantageously, the R′₁ and R′₂ groups represent a caprilyl chain, the di-alkyl carbonate used being therefore di-caprilyl carbonate.

The di-alkyl carbonate preferably represents from 0.02 to 2.5%, more preferably from 0,1 to 1% in weight based on the total weight of the composition.

Thus, the composition according to the invention could contain from 0.32 to 19% in weight, and preferably from 2.1 to 9% in weight of the quaternary associative complex formed by the four above-identified compounds.

In addition to the four previously described compounds, the composition according to the invention comprises at least one alkaline agent, at least one dye precursor and at least one oxidizing compound.

The alkaline agent(s) present in the composition according to the invention could be selected from the group consisting of aminomethylpropanol, aqueous ammonia, monoethanolamine, diethanolamine, triethanolamine and the mixtures thereof.

The alkaline agent(s) preferably represent(s) from 1 to 30%, more preferably from 1 to 4% in weight based on the total weight of the composition. More particularly, the composition according to the invention advantageously comprises from 1 to 4% in weight of aqueous ammonia.

The oxidation dye precursors present in the composition according to the invention are generally aromatic compounds such as aromatic diamines, aminophenols or phenols. They could also be selected amongst bases and coupling agents.

The bases or primary intermediates could be aromatic amines, diaminophenols or aminophenols wherein the NH₂ and OH groups are in the ortho or para position one with respect to the others. Examples are for example para-phenylene diamine (pPD), ortho-aminophenol (oAP), para-methylamino phenol (pMAP), para-amino phenol (pAP), para-toluene diamine (pTD) and N-phenyl paraphenylene diamine (NpPD).

The coupling agents or modifiers could be meta-diamines, meta-aminophenols, polyphenols or naphtols. Examples are meta-aminophenol (mPD), resorcinol (R), 1-naphthol (1-N), meta-phenylene diamine (mPD), para-amino ortho-cresol (pAOC), 1,5-dihydroxynaphthalene (1,5-DHN) and/or 2,7-dihydroxynaphthalene (2,7-DHN).

The dye precursor(s) preferably represent(s) from 0.5 to 10%, more preferably from 1 to 2.5% in weight based on the total weight of the composition.

The composition according to the invention finally comprises at least one oxidizing compound. A particularly preferred oxidizing agent is hydrogen peroxide.

Advantageously, the oxidizing agent(s) is/are introduced in the composition according to the invention as a solution, with a content preferably ranging from 1.5 to 12%, more preferably from 6 to 9%, in weight based on the weight of the solution. In such a case, the solution preferably represents 50% in weight based on the total weight of the composition. Thus, the oxidizing compound content in the composition according to the invention preferably ranges from 0.75 to 6%, more preferably from 3 to 4.5% in weight based on the total weight of the composition.

The pH of the composition preferably ranges from 7 to 11, more preferably from 8.5 to 10.5.

Thus, advantageously, the composition according to the present invention comprises:

from 0.1 to 10% in weight of a protein hydrolyzate, preferaby a sericin hydrolyzate,

from 0.1 to 1.5% in weight of a quaternized copolymer of di-methylallyl ammonium and acrylic acid,

from 0.1 to 5% in weight of N-methyl triethanolamine methylsulphate di-alkyl ester,

from 0.02 to 2.5% in weight of di-alkyl carbonate,

from 0.75 to 6% in weight of at least one oxidizing compound,

from 0.5 to 10% in weight of at least one dye precursor, and

a sufficient amount of at least one alkaline agent for reaching a pH ranging from 7 to 11, preferably from 8.5 to 10.5.

More preferably, the composition according to the present invention comprises:

from 1 to 5% in weight of a protein hydrolyzate, preferaby of a sericin hydrolyzate,

from 0.5 to 1% in weight of a quaternized copolymer of di-methylallyl ammonium and acrylic acid,

from 0.5 to 2% in weight of N methyl triethanolamine methylsulphate di-alkyl ester,

from 0,1 to 1% in weight of di-alkyl carbonate,

from 3 to 4.5% in weight of at least one oxidizing compound,

from 1 to 2.5% in weight of at least one dye precursor, and

a sufficient amount of at least one alkaline agent for reaching a pH ranging from 7 to 11, preferably from 8.5 to 10.5.

The composition according to this invention could further comprise various adjuvants usually present in oxidation dye compositions. Such adjuvants could be naturally occurring or synthetic fatty acids, natural occurring or synthetic fatty alcohols, either oxyethylenated or polyglycerolated, mineral or vegetable oils, antioxidizing agents, sequestering agents.

The fatty acids useful as adjuvants in the composition according to this invention generally comprise from 10 to 18 carbon atoms, and preferably represents from 1 to 20%, more preferably from 5 to 15% in weight based on the total weight of the composition.

The fatty alcohols useful as adjuvants in the composition according to the invention could be selected amongst oleic alcohol, lauric alcohol, myristic alcohol, cetyl alcohol, stearyl alcohol, ceteareth 33 and mixtures thereof. They preferably represent from 1 to 25%, more preferably from 5 to 20% in weight based on the total weight of the composition.

An example of an oil useful as an adjuvant in the composition according to this invention is copra oil.

Examples of antioxidizing agents useful as adjuvant in the composition according to this invention include sodium metabisulfite and 1-phenyl-3-methyl-5-pyrazolone.

The composition according to the invention is generally under the form of a cream or a gel, preferably under the form of a cream.

The composition of the present invention could be prepared through mixing, the various components that it comprises as is known to the man of the art.

Another object of this invention is a quaternary associative complex for dyeing keratinous fibres, comprising:
a protein hydrolyzate, preferably a sericin hydrolyzate,
a quaternized copolymer of di-methylallyl ammonium and acrylic acid,
a N-methyl triethanolamine methylsulphate di-alkyl ester, and
a di-alkyl carbonate.

Another object of this invention is also a base, having the aspect of a gel or a cream, for preparing a composition such as previously defined, comprising:
a protein hydrolyzate, preferably a sericin hydrolyzate,
a quaternized copolymer of di-methylallyl ammonium and acrylic acid,
a N-methyl triethanolamine methylsulphate di-alkyl ester, and
a di-alkyl carbonate,
at least one dye precursor, and
at least one alkaline agent.

Another object of this invention is to provide a solution for preparing a composition such as previously defined, comprising at least one oxidizing compound, preferably hydrogen peroxide.

Advantageously, the oxidizing compound(s) is/are present in the solution with a content preferably ranging from 1.5 to 12%, more preferably from 6 to 9%, in weight based on the weight of the solution.

Another object of the invention is to provide a method for revealing the dyeing of a base such as previously defined, comprising the step involving mixing the previously defined base with the previously defined solution, preferably in a 1/1 weight ratio.

Finally, the invention also relates to a method for dyeing keratinous fibres, preferably hair, comprising the following steps of:
applying onto said fibres the composition according to the invention, for a period of time sufficient for obtaining the desired colouring, generally from 10 to 45 minutes, preferably in the order of 30 minutes,
rinsing and drying the fibres.

The composition according to the invention allows to dye hair with a covering power and a durability higher than those of the commonly used compositions. The colorings perfectly and uniformly cover the hair with no overload on the sensitized areas and show a considerably improved shampoo stability. The shades keep their brightness and their highlights, more intensely on the dye day, and do not lose their radiance shampoo after shampoo.

A hypothesis that could be formulated, without the Applicant being related to it, is that the formed complex improves the penetration and attachment of dyeing agents in the fibre. In addition, because of their cationic character, the compounds included in the composition according to the invention show a strong affinity to keratin and form at the fibre surface a film that could have a protective and repairing role during the oxidative reaction.

When protected by the composition according to the invention, the hair holds after the dyeing operation a soft and silky touch. It keeps the flexibility, the strength, the elasticity and the brightness of healthy and natural hair.

The moisturizing and soothing properties of the peptidic fractions of the complex also have the advantage to significantly limit the diversity and intensity of the cutaneous discomfort reactions generally related to the application of oxidative dyeing products.

This invention will be better understood from the following non limiting examples.

### EXAMPLE 1

Preparation of a Permanent Dyeing Cream with a Chestnut Brown Shade

A dyeing composition according to the invention is formulated from a base and a solution comprising an oxidizing compound.

The formula of the base is as follows, expressed in grams:

| | |
|---|---|
| Stearyl cetyl alcohol | 9 |
| Cetearyl alcohol/Ceteareth 33 | 11.5 |
| Oleic acid | 5 |
| Copra oil | 1.5 |
| Sodium metabisulfite | 0.6 |
| Pentenic acid sodium salt | 1 |
| Sericin hydrolyzate (SETAKOL, marketed by | 3 |
| PENTAPHARM company) | |
| Di n-octyl carbonate | 1 |
| Di-cocoylethyl Hydroxyethylmonium | 1.5 |
| Methylsulphate | |
| (DEHYQUART L80, marketed by COGNIS | |
| company) | |
| Acrylic/dimethyl diallyl ammonium chloride | 1.5 |
| copolymer (MERQUAT 280 (polyquaternium 22) | |
| marketed by ONDEO NALCO company) | |
| Aqueous ammonia | 12 |
| Meta-phenylene diamine | 0.04 |
| Resorcinol | 0.6 |
| Para-phenylene diamine | 0.5 |
| Para-amino phenol | 0.07 |
| Meta-amino phenol | 0.09 |
| 1-phenyl-3-methyl-5-pyrazolone | 0.1 |
| Fragrance | 0.4 |
| Demineralized water | qsp 100 |

In use, such a base is diluted with 100 g of aqueous hydrogen peroxide volumes (i.e. 6% in weight of hydrogen peroxide), so as to give a dyeing composition according to the invention being then applied to the hair for 30 minutes.

### EXAMPLE 2

Preparation of a Permanent Dyeing Cream with a Fair Shade

A dyeing composition according to this invention is formulated from a base and a solution comprising an oxidizing compound.

The formula of the base is as follows, expressed in grams:

| | |
|---|---|
| Stearyl cetyl alcohol | 9 |
| Cetearyl alcohol/Ceteareth 33 | 11.5 |
| Oleic acid | 5 |
| Copra oil | 1.5 |
| Sodium metabisulfite | 0.6 |
| Pentenic acid sodium salt | 1 |
| Sericin hydrolyzate (SETAKOL, marketed by | 3 |
| PENTAPHARM company) | |
| Di n-octyl carbonate | 1 |
| Di-cocoylethyl Hydroxyethylmonium | 1.5 |
| Methylsulphate | |
| (DEHYQUART L80, marketed by COGNIS | |
| company) | |
| Acrylic/dimethyl diallyl ammonium chloride | 1.5 |
| copolymer (MERQUAT 280 (polyquaternium 22) | |
| marketed by ONDEO NALCO company) | |
| Aqueous ammonia | 12 |
| Resorcinol | 0.2 |
| Para-phenylene diamine | 0.15 |
| Para-amino phenol | 0.18 |
| Meta-amino phenol | 0.08 |
| 1-phenyl-3-methyl-5-pyrazolone | 0.1 |
| Ortho-aminophenol | 0.03 |
| NNB HE para phenylene diamine sulfate | 0.1 |
| Fragrance | 0.4 |
| Demineralized water | qsp 100 |

In use, such a base is diluted with 100 g of aqueous hydrogen peroxide at 20 volumes, so as to give a dyeing composition according to the invention being then applied to the hair for 30 minutes.

### EXAMPLE 3

Shampoo Resistance of the Dyeing Composition According to the Invention

The shampoo resistance of the dyeing composition is quantified through measurement of the colour variation generated by six successive shampoos on wool samples being subjected to a dyeing operation (light brown shade) using:
a composition according to the invention containing a quaternary associative complex, or
a composition which does not contain any quaternary associative complex (i.e. that does not contain any of the 4 components of the quaternary associative complex), or
a composition containing a binary association of active ingredients A, or
a composition containing a binary association of active ingredients B.

Each composition is formulated from a dyeing base and a solution comprising an oxidizing compound.

The formulations of the 4 dyeing bases to be used are as follows:

Dyeing Base According to the Invention with a Quaternary Associative Complex

| | |
|---|---|
| Stearyl cetyl alcohol | 9 |
| Cetearyl alcohol/Ceteareth 33 | 11.5 |
| Oleic acid | 5 |
| Copra oil | 1.5 |
| Sodium metabisulfite | 0.6 |
| Pentenic acid sodium salt | 1 |
| Sericin hydrolyzate (SETAKOL, marketed by | 3 |
| PENTAPHARM company) | |
| Di n-octyl carbonate | 1 |
| Di-cocoylethyl Hydroxyethylmonium | 1.5 |
| Methylsulphate | |
| (DEHYQUART L80, marketed by COGNIS | |
| company) | |
| Acrylic/dimethyl diallyl ammonium chloride | 1.5 |
| copolymer (MERQUAT 280 (polyquaternium 22) | |
| marketed by ONDEO NALCO company) | |
| Aqueous ammonia | 12 |
| Meta-phenylene diamine | 0.035 |
| Resorcinol | 0.5 |
| Para-phenylene diamine | 0.4 |
| Para-amino phenol | 0.05 |
| Meta-amino phenol | 0.07 |
| 1-phenyl-3-methyl-5-pyrazolone | 0.1 |
| Fragrance | 0.4 |
| Demineralized water | qsp 100 |

Dyeing Base Without any Quaternary Associative Complex

| | |
|---|---|
| Stearyl cetyl alcohol | 9 |
| Cetearyl alcohol/Ceteareth 33 | 11.5 |
| Oleic acid | 5 |
| Copra oil | 1.5 |
| Sodium metabisulfite | 0.6 |
| Pentenic acid sodium salt | 1 |
| Aqueous ammonia | 12 |
| Meta-phenylene diamine | 0.035 |
| Resorcinol | 0.5 |
| Para-phenylene diamine | 0.4 |
| Para-amino phenol | 0.05 |
| Meta-amino phenol | 0.07 |
| 1-phenyl-3-methyl-5-pyrazolone | 0.1 |
| Fragrance | 0.4 |
| Demineralized water | qsp 100 |

Dyeing Base With a Binary Association A

| | |
|---|---|
| Stearyl cetyl alcohol | 9 |
| Cetearyl alcohol/Ceteareth 33 | 11.5 |
| Oleic acid | 5 |
| Copra oil | 1.5 |
| Sodium metabisulfite | 0.6 |
| Pentenic acid sodium salt | 1 |
| Sericin hydrolyzate (SETAKOL, marketed by | 3 |
| PENTAPHARM company) | |
| Acrylic/dimethyl diallyl ammonium chloride | 1.5 |
| copolymer (MERQUAT 280 (polyquaternium | |
| 22) marketed by ONDEO NALCO company) | |
| Aqueous ammonia | 12 |
| Meta-phenylene diamine | 0.035 |
| Resorcinol | 0.5 |
| Para-phenylene diamine | 0.4 |
| Para-amino phenol | 0.05 |
| Meta-amino phenol | 0.07 |
| 1-phenyl-3-methyl-5-pyrazolone | 0.1 |
| Fragrance | 0.4 |
| Demineralized water | qsp 100 |

Dyeing Base With a Binary Association B

| | |
|---|---|
| Stearyl cetyl alcohol | 9 |
| Cetearyl alcohol/Ceteareth 33 | 11.5 |
| Oleic acid | 5 |
| Copra oil | 1.5 |
| Sodium metabisulfite | 0.6 |
| Pentenic acid sodium salt | 1 |
| Di n-octyl carbonate | 1 |
| Di-cocoylethyl Hydroxyethylmonium | 1.5 |
| Methylsulphate (DEHYQUART L80, marketed the | |
| COGNIS company) | |
| Aqueous ammonia | 12 |
| Meta-phenylene diamine | 0.035 |
| Resorcinol | 0.5 |
| Para-phenylene diamine | 0.4 |
| Para-amino phenol | 0.05 |
| Meta-amino phenol | 0.07 |
| 1-phenyl-3-methyl-5-pyrazolone | 0.1 |
| Fragrance | 0.4 |
| Demineralized water | qsp 100 |

In use, each dyeing base is diluted with 100 g of aqueous hydrogen peroxide at 20 volumes, so as to give a dyeing composition, being then applied to the wool samples for 30 minutes.

The measurements, in L*a* b* colour space, are made using a MINOLTA CR 210 colorimeter.

The colour durability is quantified by the value of the colour deviation ΔE*ab, as defined by the following equation: ΔE*ab=[(ΔL*)²+(Δa*)²+(Δb*)²]^{1/2}.

The decrease in value ΔE is proportional to the improvement in the colour durability.

The obtained results are given in table I.

TABLE I

| | ΔE |
|---|---|
| COMPOSITION | after 6 shampoos |
| Without quaternary associative complex | 12.81 |
| With quaternary associative complex | 5.61 |
| With binary association A | 10.69 |
| With binary association B | 10.4 |

After 6 shampoos, the wool fabrics as dyed by the composition that do not contain the quaternary associative complex have a colour variation of 12.81 as compared with 5.61 only for the fabrics dyed with the composition according to the invention containing the complex.

The presence of the quaternary associative complex therefore improves the dye strength to washing.

In comparison, the binary association of components of the quaternary associative complex improves the dye resistance to removal through shampoos, but much less significantly than in the case of the quaternary complex.

There is accordingly a true synergism between the four active ingredients constituting the quaternary associative complex according to the invention.

### EXAMPLE 4

Resistance to the Action of Ultraviolet Rays

The resistance of the dye to ultraviolet rays is quantified by the measurement of the colour variation (ΔE) generated by the exposure to a intense irradiation of wool samples being subjected to a dye (light brown shade) using:
a composition according to the invention containing a quaternary associative complex, or
a composition which does not contain any quaternary associative complex (i.e. that does not contain any of the 4 components of the quaternary associative complex), or
a composition containing a binary association of active ingredients A, or
a composition containing a binary association of active ingredients B.

The formulations of such compositions are identical to those of the dyeing compositions used in example 3.

The measurements, in L*a* b* colour space, are made using a MINOLTA CR 210 calorimeter.

The irradiation is carried out using a SUNTEST device, the wool samples being subjected to a intense light ray for 40 hours.

The obtained results are given in table II.

TABLE II

| | ΔE |
|---|---|
| COMPOSITION | after 6 shampoos |
| Without quaternary associative complex | 8.17 |
| With quaternary associative complex | 5.52 |
| With binary association A | 8.01 |
| With binary association B | 7.4 |

The presence of the quaternary associative complex makes it possible to significantly improve the colour stability under an ultraviolet irradiation.

The synergism of the four active ingredients is against evidenced the colouring having a better stability in the presence of the four components of the quaternary associative complex according to the invention.

### EXAMPLE 5

Protective Effect of the Quaternary Associative Complex

The protective effect of the quaternary associative complex is quantified through the measurement of mechanical properties (elasticity modulus at 5% of elongation) of the hair after dyeing using:

a composition according to the invention containing a quaternary associative complex, or

a composition according to the invention which does not contain any quaternary associative complex (i.e. that does not contain any of the 4 components of the quaternary associative complex).

The formulations of those both compositions are identical to both corresponding compositions from example 3.

The measurements are made using a LLOYD LRY DYNAMOMETER connected to a computer.

The elasticity modulus of a hair lock is obtained measuring the strength/elongation curve slope (in Newton/elongation %). The ratio of the two moduli measured in the presence and in the absence of the complex makes it possible to evaluate the effect of said complex on the mechanical properties of the hair.

The results are given in Table III.

TABLE III

| | Elasticity modulus | |
|---|---|---|
| Composition | (Newton/5% elongation) | Improvement % |
| Without quaternary | 3.41 | 7 |
| associative complex | | |
| With quaternary | 3.19 | |
| associative complex | | |

The modulus values are significantly different at risk 5%. The quaternary complex allows for improving by 7% the elastic properties of the hair after dyeing with the composition according to the invention, showing the protective properties of the quaternary associative complex according to the invention.

1. A composition for permanent dyeing of keratinous fibres, comprising at least one dye precursor, at least one alkaline agent, and at least one oxidizing compound, characterized in that it further comprises:
a protein hydrolyzate,
a quaternized copolymer of di-methylallyl ammonium and acrylic acid,
a N-methyl triethanolamine methylsulphate di-alkyl ester, and
a di-alkyl carbonate.

2. A composition according to claim 1, characterized in that the protein hydrolyzate, the quaternized copolymer of dimethylallyl ammonium and acrylic acid, the N-methyl triethanolamine methylsulphate di-alkyl ester and the di-alkyl carbonate form a quaternary associative complex within the composition.

3. A composition according to claim 1, characterized in that the protein hydrolyzate is a sericin hydrolyzate.

4. A composition according to claim 1, characterized in that the quaternized copolymer of di-methylallyl ammonium and acrylic acid meets the following formula I: 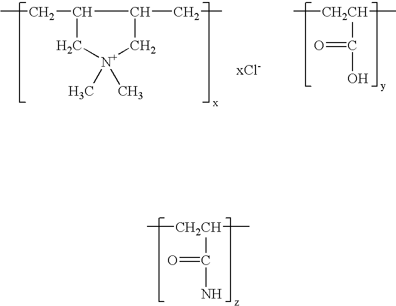
wherein x ranges from 1 to 1,000, preferably from 1 to 100, and y and z independently range from 0 to 1,000, preferably from 0 to 100.

5. A composition according to claim 1, characterized in that the quaternized copolymer of di-methylallyl ammonium and acrylic acid is a polyquaternium, preferably a polyquaternium 22.

6. A composition according to claim 1, characterized in that the N methyl triethanolamine methylsulphate di-alkyl ester meets the following formula II: 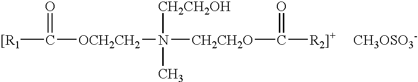
wherein R₁ and R₂ are Cₙ alkyl or alkenyl groups, wherein n ranges from 1 to 100, preferably from 6 to 20.

7. A composition according to claim 6, characterized in that R₁ and R₂ are selected amongst caproyl, caprylyl, capryl, lauryl, myristyl, cetyl, stearyl, arachidyl, behenyl, caproleyl, lauroleyl, myristoleyl, palmitolyl, oleyl, linoleyl, linolenyl, arachidonyl et erucyl groups.

8. A composition according to claim 1, characterized in that the N-methyl triethanolamine methylsulphate di-alkyl ester is a N-methyl triethanolamine methylsulphate dicocoyl ester.

9. A composition according to claim 1, characterized in that the di-alkyl carbonate meets the following formula III 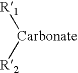
wherein R′₁ and R′₂ are Cₙ, straight or branched, alkyl or alkenyl, where n ranges from 1 to 100, preferably from 6 to 20.

10. A composition according to claim 9, characterized in that R′₁ and R′₂ are selected amongst caproyl, capryilyl, capryl, lauryl, myristyl, cetyl, stearyl, arachidyl, behenyl, caproleyl, lauroleyl, myristoleyl, palmitoleyl, oleyl, linoleyl, linolenyl, arachidonyl et erucyl groups.

11. A composition according to claim 10, characterized in that the di-alkyl carbonate is di-caprilyl carbonate.

12. A composition according to claim 1, characterized in that the oxidizing compound is hydrogen peroxide.

13. A composition according to claim 1, characterized in that the alkaline agent(s) is/are selected from the group consisting of aminomethylpropanol, monoethanolamine, diethanolamine, triethanolamine, ammonia and the mixtures thereof.

14. A composition according to claim 1, characterized in that the dye precursors are selected amongst aromatic diamines, aminophenols and phenols.

15. A composition according to claim 14, characterized in that the dye precursors are selected amongst para-phenylene diamine (pPD), ortho-aminophenol (oAP), para-methylamino phenol (pMAP), para-amino phenol (pAP), para-toluene diamine (pTD), N-phenyl paraphenylene diamine (NpPD), meta-aminophenol (mPD), resorcinol (R), 1-naphthol(1-N), meta-phenylene diamine (mPD), para-amino ortho-cresol (pAOC), 1,5-dihydroxynaphthalene (1,5-DHN) and/or 2,7 dihydroxynaphtalene (2,7-DHN).

16. A composition according to claim 1, characterized in that its pH ranges from 7 to 11, preferably from 8.5 to 10.5.

17. A composition according to claim 1, characterized in that the protein hydrolyzate represents from 0.1 to 10%, more preferably from 1 to 5% in weight based on the total weight of the composition.

18. A composition according to claim 1, characterized in that the quaternized copolymer of di-methylallyl ammonium and acrylic acid represents from 0.1 to 1.5%, more preferably from 0.5 to 1%, in weight based on the total weight of the composition.

19. A composition according to claim 1, characterized in that N-methyl triethanolamine methylsulphate di-alkyl ester represents from 0.1 to 5%, more preferably from 0.5 to 2%, in weight based on the total weight of the composition.

20. A composition according to claim 1, characterized in that the di-alkyl carbonate represents from 0.02 to 2.5%, preferably from 0.1 to 1% in weight of the total weight of the composition.

21. A composition according to claim 1, characterized in that the oxidizing compound(s) represent(s) from 0.75 to 6%, preferably from 3 to 4.5% in weight, based on the total weight of the composition.

22. A composition according to claim 1, characterized in that the alkaline agent(s) represent(s) from 1 to 30%, preferably from 1 to 4% in weight based on the total weight of the composition.

23. A composition according to claim 1, characterized in that the dye precursor(s) represent(s) from 0.5 to 10%, preferably from 1 to 2.5% in weight based on total weight of the composition.

24. A composition according to claim 1, characterized in that it comprises:
from 0.1 to 10% in weight of a protein hydrolyzate, preferaby a sericin hydrolyzate,
from 0.1 to 1.5% in weight of a quaternized copolymer of di-methylallyl ammonium and acrylic acid,
from 0.1 to 5% in weight of N-methyl triethanolamine methylsulphate di-alkyl ester,
from 0.02 to 2,5% in weight of di-alkyl carbonate,
from 0.75 to 6% in weight of at least one oxidizing compound,
from 0.5 to 10% in weight of at least one dye precursor, and
a sufficient amount of at least one alkaline agent for reaching a pH ranging from 7 to 11, preferably from 8.5 to 10.5.

25. A composition according to claim 1, characterized in that it comprises:
from 1 to 5% in weight of a protein hydrolyzate, preferably a sericin hydrolyzate,
from 0.5 to 1% in weight of a quaternized copolymer of di-methylallyl ammonium and acrylic acid,
from 0.5 to 2% in weight of N-methyl triethanolamine methylsulphate di-alkyl ester,
from 0.1 to 1% in weight of di-alkyl carbonate,
from 3 to 4.5% in weight of at least one oxidizing compound,
from 1 to 2.5% in weight of at least one dye precursor, and
a sufficient amount of at least one alkaline agent for reaching a pH ranging from 7 to 11, preferably from 8.5 to 10.5.

26. A composition according to preceding claim 1, characterized in that it comprises adjuvants selected amongst naturally occurring or synthetic fatty acids, naturally occurring or synthetic fatty alcohols, either oxyethylenated or polyglycerolated, mineral or vegetable oils, antioxidizing agents, sequestering agents.

27. A composition according to claim 26, characterized in that the fatty acids represent from 1 to 20%, preferably from 5 to 15% in weight based on the total weight of the composition.

28. A composition according to claim 26, characterized in that the fatty acids represent from 1 to 25%, preferably from 5 to 20% in weight based on the total weight of the composition.

29. A composition according to claim 1, characterized in that it is in the form of a cream or a gel, preferably in the form of a cream.

30. A quaternary associative complex for dyeing keratinous fibres, characterized in that it comprises:
a protein hydrolyzate, preferably a sericin hydrolyzate,
a quaternized copolymer of di-methylallyl ammonium and acrylic acid,
a N-methyl triethanolamine methylsulphate di-alkyl ester, and
a di-alkyl carbonate.

31. A base, having the form of a gel or a cream, for preparing a composition according to claim 1, characterized in that it comprises:
a protein hydrolyzate, preferably a sericin hydrolyzate,
a quaternized copolymer of di-methylallyl ammonium and acrylic acid,
a N-methyl triethanolamine methylsulphate di-alkyl ester, and
a di-alkyl carbonate.
at least one dye precursor, and
at least one alkaline agent.

32. A method for revealing the composition dye according to claim 1, characterized in that it comprises a step involving mixing the base such as defined in claim 31 with a solution comprising at least one oxidizing compound, preferably hydrogen peroxide.

33. A method according to claim 32, characterized in that the oxidizing compound(s) represent(s) from 1.5 to 12%, preferably from 6 to 9%, in weight based on the weight of said solution.

34. A method according to claim 32 characterized in that said base and said solution are mixed in a 1/1 weight ratio.

35. A method for dyeing keratinous fibres, preferably hair, comprising the following steps of:
applying onto said fibres a composition according to claim 1, for a period of time sufficient for obtaining the desired colouring, preferably from 10 to 45 minutes, more preferably in the order of 30 minutes,
rinsing and drying of the fibres.
